(19) United States
(12) Patent Application Publication
(10) Pub. No.: US 2017/0015994 A1
(43) Pub. Date: Jan. 19, 2017

(54) METHODS FOR IN VIVO GENOME EDITING

(71) Applicant: MASSACHUSETTS INSTITUTE OF TECHNOLOGY, Cambridge, MA (US)

(72) Inventors: Daniel G. Anderson, Framingham, MA (US); Hao Yin, Cambridge, MA (US); Roman L. Bogorad, Cambridge, MA (US); Tyler E. Jacks, Newton, MA (US); Wen Xue, Cambridge, MA (US)

(21) Appl. No.: 15/121,292

(22) PCT Filed: Feb. 24, 2015
(86) PCT No.: PCT/US2015/017246

§ 371 Date: Aug. 24, 2016

Related U.S. Application Data
(60) Provisional application No. 61/943,934, filed on Feb. 24, 2014.

(51) Int. Cl. C12N 15/10; A61K 31/713; A61K 9/00

(52) U.S. Cl. CPC C12N 15/1024; A61K 9/0019; A61K 31/713

(57) ABSTRACT

Methods of transfecting cells in vivo, by administering an injectable pharmaceutical composition including a genome editing composition and a pharmaceutically acceptable carrier to a subject by hydrodynamic injection into a vessel of the subject are disclosed. Typically, the pharmaceutical composition is administered in a volume and at rate of injection suitable to transfect target eukaryotic cells in the subject with an effective amount of the genome editing composition to alter the genome of the target cells. In preferred embodiments the subject is a mammal, such as rodent, or a primate such as a human. The methods can be used to treat one or more symptoms of a genetic disease or condition.

### STATEMENT REGARDING FEDERALLY SPONSORED RESEARCH

This invention was made with government support under grants 2-PO1-CA42063, P30-CA14051, 1K99CA169512, and 5-U54-CA151884-04 awarded by the National Institutes of Health. The government has certain rights in the invention.

### FIELD OF THE INVENTION

The invention is generally directed to compositions and methods for in vivo genome editing.

### BACKGROUND OF THE INVENTION

Precise genome modification holds great promise to treat genetic diseases. The RNA-guided type II bacterial CRISPR/Cas system (Jinek, et al., Science, 337, 816-821 (2012). Sternberg, et al., “DNA interrogation by the CRISPR RNA-guided endonuclease Cas9”, Nature advance online publication (2014).) has been engineered into a powerful genome editing tool containing a human-codon optimized Cas9 and a single-guide RNA (sgRNA) (Cong, L. et al., Science, 339, 819-823 (2013), Mali, P. et al., Science, 339, 823-826 (2013), Hsu, et al., Nat. Biotechnol., 31, 827-832 (2013), Jinek, et al., Elife 2, e00471 (2013), Cho, et al., Nat. Biotechnol., 31, 230-232 (2013)). The sgRNA targets the Cas9 nuclease to the complementary 20 nucleotide (nt) genomic region harboring a 5′-NGG-3′ protospacer-adjacent motif (PAM). The double-stranded DNA breaks generated by Cas9 are repaired by nonhomologous end-joining (NHEJ) or homology-directed repair (HDR).

CRISPR-mediated genome editing has been applied to a wide variety of organisms, such as bacteria, yeast, C. elegans, Drosophila, plants, zebrafish, and mouse and human cells (reviewed in Mali, et al., Nat Methods, 10, 957-963 (2013)). In rodent and primate zygotes, CRISPR can efficiently generate multiplexed mutant alleles or reporter genes (Wang, et al., Cell, 153, 910-918 (2013), Yang, et al., Cell, 154, 1370-1379 (2013), Li, et al., Nat. Biotechnol., 31, 684-686 (2013), Li, et al., Nat. Biotechnol., 31, 681-683 (2013), Shen, et al., Cell Res., 23, 720-723 (2013), Niu, et al., “Generation of Gene-Modified Cynomolgus Monkey via Cas9/RNA-Mediated Gene Targeting in One-Cell Embryos”, Cell (2014).).

Nickase version of Cas9 and off-set sgRNAs have been used to reduce off-target effects (Mali, et al., Nat. Biotechnol., 31, 833-838 (2013), Ran, et al., Cell, 154, 1380-1389 (2013)). SgRNA genome-wide library screens can be done in human cells (Shalem, et al., “Genome-Scale CRISPR/Cas9 Knockout Screening in Human Cells,” Science (2013), Wang, et al., “Genetic Screens in Human Cells Using the CRISPR/Cas9 System”, Science (2013)). Correction of genetic disease genes has been demonstrated in organoids (Schwank, et al., Cell Stem Cell, 13, 653-658 (2013)) and mouse zygotes (Wu, et al., Cell Stem Cell, 13, 659-662 (2013)), and although zinc finger nuclease delivered by viral vectors was used to correct hemophilia in mice (Li, et al., Nature, 475, 217-221 (2011)), administration of Cas9/sgRNA in adult mammalian organs to correct genetic disease genes in vivo has not been reported. Therefore, there remains a need for compositions and methods for carrying out genome editing in vivo, particularly CRISPR-mediated genome editing, in mammals.

Therefore, it is an object of the invention to provide compositions and methods of carrying out genome editing in vivo.

It is a further object of the invention to provide compositions and methods for carrying out CRISPR/Cas-mediated genome editing in vivo.

It is a further object of the invention to provide compositions and methods that enable genome editing, particularly CRISPR/Cas-mediated genome editing, in an effective amount to reduce one or more symptoms of genetic disease.

### SUMMARY OF THE INVENTION

Methods of transfecting cells in vivo with genome editing compositions are disclosed. The methods typically include administering to a subject an injectable pharmaceutical composition including a genome editing composition and a pharmaceutically acceptable carrier by hydrodynamic injection into a blood or lymph vessel. The genome editing composition typically includes nucleic acids, for example, a plasmid or other suitable vector or expression construct that encodes the elements necessary to carry out CRISPR/Cas-mediated, zinc finger nuclease-mediated, or TALEN-mediate mediated genome editing in a cell, and, optionally, a donor polynucleotide.

Typically, the pharmaceutical composition is administered in a volume and at rate of injection suitable to transfect target eukaryotic cells in the subject with an effective amount of the genome editing composition to alter the genome of the target cells. In preferred embodiments the subject is a mammal, such as rodent, or a primate such as a human.

The methods can be used to treat one or more symptoms of a genetic disease or condition. For example, the methods can be used to correct a point mutation, such as a point mutation in a promoter, or gene intron or exon, in the genome of the target cells. The point mutation can be the cause of aberrant transcription of a gene or translation of a mutated protein in the subject.

In preferred embodiments the genetic disease is one characterized by positive selection, wherein alteration of the genome of between 1% and 75%, 10% and 50%, or 20% and 40% of the target cells is effective to alleviate one or more symptoms of the disease or condition. In one embodiment, the target cells are hepatocytes and the disease or condition is hereditary tyrosinemia type I (HTI).

In the most preferred embodiments, the genome editing is mediated by CRISPR/Cas elements. Typically, the CRISPR/Cas-mediated genome editing composition used in the disclosed methods includes one or more plasmids encoding (a) a chimeric RNA (chiRNA) polynucleotide sequence, wherein the polynucleotide sequence includes (i) a guide sequence capable of hybridizing to a genomic target sequence in the target cells, (ii) a tracr mate sequence, and (iii) a tracr sequence; and (b) an enzyme-coding sequence encoding a CRISPR enzyme, wherein (a) and (b) are operably linked to the same or different promoters capable of driving expression of (a) and (b) in the target cells in an amount effective to induce a single or double strand break at a target site in genome of the target cells.

Preferably, the CRISPR/Cas-mediated genome editing composition further includes a donor polynucleotide suitable for recombination into the genome of the target cells at or adjacent to the target site. The donor polynucleotide can be used to introduce into the target cells' genome one or more insertions, deletions, or substitution in the target cells' genome. In a preferred embodiment, the substitution corrects a point mutation, for example a point mutation associated with genetic disease or condition.

The hydrodynamic injection can result in systemic circulation of the injectable pharmaceutical composition, or region or local circulation of the pharmaceutical composition. In some embodiments, the method further includes occluding one or more vessels of the subject to direct the flow of the pharmaceutical composition toward the target cells. The hydrodynamic injection can be carried out through any suitable vessel, including, but not limited to, the tail vein, tail artery, inferior vena cava, superior vena cava, jugular vein, hepatic vein, hepatic artery, portal vein, bile duct, saphenous, cephalic and median veins, femoral vein, femoral artery, brachial and popliteal arteries, iliac arteries, renal vein, carotid artery, or aorta.

### BRIEF DESCRIPTION OF THE DRAWINGS

FIG. 1A is an illustration showing the design of an experiment to test CRISPR/Cas-mediated genome editing in vivo. Fah^{mut/mut} mice harbor a homozygous G→A point mutation at the last nucleotide of exon 8, causing splicing skipping of exon 8. pX330 plasmids expressing Cas9 and sgRNA targeting the Fah locus were hydrodynamically injected into the liver. A ssDNA oligo with “G” was co-injected to serve as a donor template to repair the “A” mutation. PAM sequences of three sgRNAs are in bold. Exon and intron sequences are in upper and lower cases respectively. FIG. 1B is a line graph showing the weight ratio (normalized to pre-injection) as function of time (days) for Fah^{mut/mut} mice injected with saline only, ssDNA oligo only, ssDNA oligo plus pX330 (empty Cas9), or pX330 expressing one of three Fah sgRNAs (FAH1, FAH2, and FAH3). An arrow indicates withdrawal of NTBC water (defined as Day 0, which is 3 days post injection). FIG. 1C is a line graph showing the weight ratio (normalized to pre-injection) as function of time (days) for Fah^{mut/mut} mice injected with FAH1 or FAH3, recovered in NTBC water, and subjected to a second round of NTBC withdrawal.

FIG. 2A is a flow chart showing the tyrosine metabolic pathway in which FAH is the last enzyme. FAH deficiency causes accumulation of toxic metabolites, such as fumarylacetoacetate (FAA). NTBC blocks the pathway upstream and rescues liver damage. FIG. 2B is the genomic sequence of Fah^{mut/mut} mice (SEQ ID NO:1). The G→A splicing mutation is highlighted. Exon 8 is underlined. FIG. 2C is the sequence of FAH sgRNAs (PAM is underlined) and oligonucleotides for cloning sgRNAs (Bbs I sites are bolded). The G→A splicing mutation is bolded/italicized. FIG. 2D is a drawing showing the CRISPR/Cas construct in the pX330 plasmid, which co-expresses the sgRNA and Cas9 (adapted from Hsu, et al., Nat Biotechnol, 31:827-832 (2013)).

FIG. 3A is a line graph showing the weight ratio (normalized to pre-injection) as function of time (days) for Fah^{mut/mut} mice injected with saline only, ssDNA oligo plus pX330 (empty Cas9), or pX330 expressing Fah sgRNA 2 (FAH2). An arrow indicates withdrawal of NTBC water (defined as Day 0, which is 3 days post injection). FIG. 3B is chart showing a summary of conditions of experimental mice in first round of NTBC withdrawal in FIGS. 1 and 3A. Fisher's exact test was performed. P<0.01. FIG. 3C is a bar graph showing the weight of control and experimental (FAH treated) mice at endpoints in the first round of NTBC withdrawal in FIGS. 1 and 3A (P<0.01 (N=5)).

FIG. 4A-4C are bar graphs showing the levels of liver damage markers (AST (IU/L) (A); ALT (IU/L) (B); Total Bilurubin (mg/dL) (C)) measured in peripheral blood from Fah^{mut/mut} mice injected with saline or ssDNA oligo only or empty Cas9 (NTBC off), or FAH (NTBC off+FAH). Fah^{mut/mut} mice on NTBC water (NTBC on) served as a control. *, p<0.01 (N≧3).

FIG. 5 is a bar graph showing the results of QPCR (relative mRNA expression levels (folds)) in liver RNA from wildtype (Fah+/+), Fah^{mut/mut}, and Fah^{mut/mut} mice injected with FAH CRISPR and ssDNA oligo (FAH1, FAH2, FAH3) performed using primers spanning exons 8 and 9. Error bars are s.d. from 3 technical replicates.

FIG. 6A-6B are bar graphs showing Fah repair rate at the genomic level determined by next-generation sequencing reads with “G” (A) and the percentage of Fah indels (B), following sequencing of the Fah genomic region in total liver genomic DNA from wildtype mice (WT) and Fahmut/mut mice injected with empty Cas9 (Mut) or FAH2 (FAH2). Error bars are s.d. (N=2).

FIG. 7A is a bar graph showing the body weight (ratio normalized to pre-injection weight) of FVB mice prior to and three months after injection with saline or Cas9 plasmids. Error bars are s.d. (N=5). FIG. 7B is a chart showing the numbers of mice showing liver hyperplasia or tumor at 3 month post injection.

FIG. 8 is a bar graph showing the % FLAG+hepatocytes (as an indicator of plasmid expression) in the livers of FVB mice hydrodynamically injected with 60 μg pX330 plasmid. mean+s.d. **, p<0.0001. ***, p<0.00001. (N=3).

### DETAILED DESCRIPTION OF THE INVENTION

### I. Definitions

As used herein, the term “modified nucleotide” or “chemically modified nucleotide” defines a nucleotide that has a chemical modification of one or more of the heterocyclic base, sugar moiety or phosphate moiety constituents.

As used herein, the term “recombinagenic” refers to a DNA fragment, oligonucleotide, peptide nucleic acid, or composition as being able to recombine into a target site or sequence or induce recombination of another DNA fragment, oligonucleotide, or composition.

As used herein, the term “eukaryote” or “eukaryotic” refers to organisms or cells or tissues derived therefrom belonging to the phylogenetic domain Eukarya such as animals (e.g., mammals, insects, reptiles, and birds), ciliates, plants (e.g., monocots, dicots, and algae), fungi, yeasts, flagellates, microsporidia, and protists.

As used herein, the term “construct” refers to a recombinant genetic molecule having one or more isolated polynucleotide sequences. Genetic constructs used for transgene expression in a host organism include in the 5′-3′ direction, a promoter sequence; a sequence encoding a gene of interest; and a termination sequence. The construct may also include selectable marker gene(s) and other regulatory elements for expression.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

As used herein, the term “vector” refers to a replicon, such as a plasmid, phage, or cosmid, into which another DNA segment may be inserted so as to bring about the replication of the inserted segment. The vectors can be expression vectors.

As used herein, the term “expression vector” refers to a vector that includes one or more expression control sequences.

As used herein, the term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

As used herein, the terms “transformed,” “transgenic,” “transfected” and “recombinant” refer to a host organism into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

As used herein, the term “endogenous” with regard to a nucleic acid refers to nucleic acids normally present in the host.

As used here, the term “heterologous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/ regulatory element of a different promoter added to enhance promoter control, or an additional control element of the same promoter. The term “heterologous” thus can also encompass “exogenous” and “non-native” elements.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

As used herein, the term “polynucleotide” generally refers to any polyribonucleotide or polydeoxribonucleotide, which may be unmodified RNA or DNA or modified RNA or DNA. Thus, for instance, polynucleotides as used herein refers to, among others, single-and double-stranded DNA, DNA that is a mixture of single-and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The term “nucleic acid” or “nucleic acid sequence” also encompasses a polynucleotide as defined above.

In addition, as used herein, polynucleotide refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia.

“Oligonucleotide(s)” typically refers to relatively short polynucleotides. Often the term refers to single-stranded deoxyribonucleotides, but it can refer as well to single-or double-stranded ribonucleotides, RNA:DNA hybrids and double-stranded DNAs, among others.

### II. Methods of Genome Editing In Vivo

It has been discovered that CRISPR/Cas-mediated genome editing can be carried out in vivo in mammals. Using the methods disclosed herein, in vivo genome editing can be induced in amounts effective to genetically modify one or more cells in a subject in need thereof In preferred embodiments, the subject is a mammal, for example a rodent or primate such as a human. In particular embodiments, the methods are used to modify an effective number of cells in vivo to induce a physiological change or treat one or more symptoms of a disease, particularly a genetic disease, in a subject in need thereof In the most preferred embodiments, genome editing is CRISPR/Cas-mediated. In other embodiments, the genome editing is mediated by Transcription activator-like effector nucleases (TALENs) or other zinc finger nucleases (ZNFs)

As describe in more detail below, the methods typically include delivering to one or more cells of a target cell type an effective amount of one or more nucleic acid constructs encoding the genetic elements needed for CRISPR, TALEN, or ZNF-based genome editing to genetically modify the genome of the one or more cells in vivo.

A. Formulations for Hydrodynamic Injection

The methods described herein typically include administering to a subject a genome editing composition to a target cell by hydrodynamic injection in an effective amount to alter the genome of the target cell. As used herein, a genome editing composition refers to the elements of a genome editing system needed to carry out genome editing by the system in a mammalian subject. Suitable systems are described in more detail below and include CRISPR/Cas, zinc finger nuclease, and TALEN based systems. The genome editing compositions typically include one or more nucleic acid constructs, for example, a plasmid or other suitable vector, that expresses the important elements of the genome modifying system when transfected into a target cell. Any of the genome editing composition can optionally include a donor polynucleotide that can be recombined into the target cell's genome at or adjacent to the target site (e.g., the site of single or double stand break induced by the nuclease).

As discussed in more detail below, the genome editing composition is formulated in a pharmaceutical composition, typically a solution that is suitable for hydrodynamic administration. In preferred embodiments, the compositions do not include a viral vector.

### 1. Genome Editing Compositions

The genome editing compositions include nucleic acids that encode an element or elements that induce a single or a double strand break in the target cell's genome, and optionally a polynucleotide.

### a. Strand Break Inducing Elements

### i. CRISPR/Cas

In preferred embodiments, the element that induces a single or a double strand break in the target cell's genome is a CRISPR/Cas system. CRISPR (Clustered Regularly Interspaced Short Palindromic Repeats) is an acronym for DNA loci that contain multiple, short, direct repetitions of base sequences. CRISPRs are often associated with cas genes which code for proteins that perform various functions related to CRISPRs. The CRISPR/Cas system functions as a prokaryotic immune system by conferring resistance to exogenous genetic elements such as plasmids and phages thereby imparting for a form of acquired immunity. Endogenous CRISPR spacers recognize and silence exogenous genetic elements in a manner similar to RNAi in eukaryotic organisms.

As used herein, CRISPR/Cas-mediated genome editing composition refers to the elements of a CRISPR system needed to carry out CRISPR/Cas-mediated genome editing in a mammalian subject. As discussed in more detail below, CRISPR/Cas-mediated genome editing compositions typically include one or more nucleic acids encoding a crRNA, a tracrRNA (or chimeric thereof also referred to a guide RNA or single guide RNA) and a Cas enzyme, preferably Cas9. The CRISPR/Cas-mediated genome editing composition can optionally include a donor polynucleotide that can be recombined into the target cell's genome at or adjacent to the target site (e.g., the site of single or double stand break induced by the Cas9).

The CRISPR/Cas system has been adapted for use as gene editing (silencing, enhancing or changing specific genes) for use in eukaryotes (see, for example, Cong, Science, 15:339(6121):819-823 (2013) and Jinek, et al., i Science, 337(6096):816-21 (2012)). By transfecting a cell with the required elements including a cas gene and specifically designed CRISPRs, the organism's genome can be cut and modified at any desired location. Methods of preparing compositions for use in genome editing using the CRISPR/Cas systems are described in detail in WO 2013/176772 and WO 2014/018423, which are specifically incorporated by reference herein in their entireties.

The methods of delivery disclosed herein are suitable for use with numerous variations on the CRISPR/Cas system.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more tracr mate sequences operably linked to a guide sequence (e.g., direct repeat-spacer-direct repeat) can also be referred to as pre-crRNA (pre-CRISPR RNA) before processing or crRNA after processing by a nuclease.

As discussed in more detail below, in some embodiments, a tracrRNA and crRNA are linked and form a chimeric crRNA-tracrRNA hybrid where a mature crRNA is fused to a partial tracrRNA via a synthetic stem loop to mimic the natural crRNA:tracrRNA duplex as described in Cong, Science, 15:339(6121):819-823 (2013) and Jinek, et al., Science, 337(6096):816-21 (2012)). A single fused crRNA-tracrRNA construct is also referred to herein as a guide RNA or gRNA (or single-guide RNA (sgRNA)). Within an sgRNA, the crRNA portion can be identified as the ‘target sequence’ and the tracrRNA is often referred to as the ‘scaffold’.

In some embodiments, one or more elements of a CRISPR system is derived from a type I, type II, or type III CRISPR system. In some embodiments, one or more elements of a CRISPR system is derived from a particular organism including an endogenous CRISPR system, such as Streptococcus pyogenes.

In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence can be any polynucleotide, such as DNA or RNA polynucleotides. In some embodiments, a target sequence is located in the nucleus or cytoplasm of a cell.

In the target nucleic acid, each protospacer is associated with a protospacer adjacent motif (PAM) whose recognition is specific to individual CRISPR systems. In the Streptococcus pyogenes CRISPR/Cas system, the PAM is the nucleotide sequence NGG. In the Streptococcus thermophiles CRISPR/Cas system, the PAM is the nucleotide sequence is NNAGAAW. The tracrRNA duplex directs Cas to the DNA target consisting of the protospacer and the requisite PAM via heteroduplex formation between the spacer region of the crRNA and the protospacer DNA.

Typically, in the context of an endogenous CRISPR system, formation of a CRISPR complex (including a guide sequence hybridized to a target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. All or a portion of the tracr sequence may also form part of a CRISPR complex, such as by hybridization to all or a portion of a tracr mate sequence that is operably linked to the guide sequence.

There are many resources available for helping practitioners determine suitable target sites once a desired DNA target sequence is identified. For example, numerous public resources, including a bioinformatically generated list of about 190,000 potential sgRNAs, targeting more than 40% of human exons, are available to aid practitioners in selecting target sites and designing the associate sgRNA to affect a nick or double strand break at the site. See also, crispr.u-psud.fr/, a tool designed to help scientists find CRISPR targeting sites in a wide range of species and generate the appropriate crRNA sequence.

In some embodiments, one or more vectors driving expression of one or more elements of a CRISPR system are introduced into a target cell such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element can be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector includes one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. In some embodiments, a vector includes an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell. In some embodiments, a vector includes two or more insertion sites, each insertion site being located between two tracr mate sequences so as to allow insertion of a guide sequence at each site. In such an arrangement, the two or more guide sequences can include two or more copies of a single guide sequence, two or more different guide sequences, or combinations of these. When multiple different guide sequences are used, a single expression construct may be used to target CRISPR activity to multiple different, corresponding target sequences within a cell. For example, a single vector can include about or more than about 1, 2, 3, 4, 5, 6, 7, 8, 9, 10, 15, 20 guide sequences. In some embodiments, about or more than about 1, 2, 3, 4, 5, 6, 7, 8, 9, 10, such guide-sequence-containing vectors may be provided, and optionally delivered to a cell.

In some embodiments, a vector includes a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), CaslO, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In some embodiments, the unmodified CRISPR enzyme has DNA cleavage activity, such as Cas9. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, a vector encodes a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. As a further example, two or more catalytic domains of Cas9 (RuvC I, RuvC II, and RuvC III) can be mutated to produce a mutated Cas9 substantially lacking all DNA cleavage activity. In some embodiments, a DlOA mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%>, 1%>, 0.1%>, 0.01%, or lower with respect to its non-mutated form.

In some embodiments, an enzyme coding sequence encoding a CRISPR enzyme is codon optimized for expression in particular cells, such as eukaryotic cells. The eukaryotic cells can be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules.

The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al., Nucl. Acids Res., 28:292 (2000). Computer algorithms for codon optimizing a particular sequence for expression in a particular host cell, for example Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a CRISPR enzyme correspond to the most frequently used codon for a particular amino acid.

In some embodiments, a vector encodes a CRISPR enzyme including one or more nuclear localization sequences (NLSs). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus.

In general, the one or more NLSs are of sufficient strength to drive accumulation of the CRISPR enzyme in a detectable amount in the nucleus of a eukaryotic cell. In general, strength of nuclear localization activity may derive from the number of NLSs in the CRISPR enzyme, the particular NLS(s) used, or a combination of these factors.

Detection of accumulation in the nucleus may be performed by any suitable technique. For example, a detectable marker may be fused to the CRISPR enzyme, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g., assay for DNA cleavage or mutation at the target sequence, or assay for altered gene expression activity affected by CRISPR complex formation and/or CRISPR enzyme activity), as compared to a control no exposed to the CRISPR enzyme or complex, or exposed to a CRISPR enzyme lacking the one or more NLSs.

In some embodiments, one or more of the elements of CRISPR system are under the control of an inducible promoter, which can include inducible Cas, such as Cas9.

Cong, Science, 15:339(6121):819-823 (2013) reported heterologous expression of Cas9, tracrRNA, pre-crRNA (or Cas9 and sgRNA) can achieve targeted cleavage of mammalian chromosomes. Therefore, CRISPR system utilized in the methods disclosed herein can be encoded within a vector system which can include one or more vectors which can include a first regulatory element operably linked to a CRISPR/Cas system chimeric RNA (chiRNA) polynucleotide sequence, wherein the polynucleotide sequence includes (a) a guide sequence capable of hybridizing to a target sequence in a eukaryotic cell, (b) a tracr mate sequence, and (c) a tracr sequence; and a second regulatory element operably linked to an enzyme-coding sequence encoding a CRISPR enzyme which can optionally include at least one or more nuclear localization sequences. Elements (a), (b) and (c) can arranged in a 5′ to 3 orientation, wherein components I and II are located on the same or different vectors of the system, wherein when transcribed, the tracr mate sequence hybridizes to the tracr sequence and the guide sequence directs sequence-specific binding of a CRISPR complex to the target sequence, and wherein the CRISPR complex can include the CRISPR enzyme complexed with (1) the guide sequence that is hybridized to the target sequence, and (2) the tracr mate sequence that is hybridized to the tracr sequence, wherein the enzyme coding sequence encoding the CRISPR enzyme further encodes a heterologous functional domain. In some embodiment, one or more of the vectors encodes also encodes a suitable Cas enzyme, for example, Cas9. The different genetic elements can be under the control of the same or different promoters.

While the specifics can be varied in different engineered CRISPR systems, the overall methodology is similar. A practitioner interested in using CRISPR technology to target a DNA sequence (identified using one of the many available online tools) can insert a short DNA fragment containing the target sequence into a guide RNA expression plasmid. The sgRNA expression plasmid contains the target sequence (about 20 nucleotides), a form of the tracrRNA sequence (the scaffold) as well as a suitable promoter and necessary elements for proper processing in eukaryotic cells. Such vectors are commercially available (see, for example, Addgene). Many of the systems rely on custom, complementary oligos that are annealed to form a double stranded DNA and then cloned into the sgRNA expression plasmid. Co-expression of the sgRNA and the appropriate Cas enzyme from the same or separate plasmids in transfected cells results in a single or double strand break (depending of the activity of the Cas enzyme) at the desired target site. An exemplary vector encoding an sgRNA and a Cas9 enzyme is described in the Examples below and illustrated in FIGS. 2A-2D.

### ii. Zinc Finger Nucleases

In some embodiments, the element that induces a single or a double strand break in the target cell's genome is a nucleic acid construct or constructs encoding a zinc finger nucleases (ZFNs). ZFNs are typically fusion proteins that include a DNA-binding domain derived from a zinc-finger protein linked to a cleavage domain.

The most common cleavage domain is the Type IIS enzyme Fok I. Fok I catalyzes double-stranded cleavage of DNA, at 9 nucleotides from its recognition site on one strand and 13 nucleotides from its recognition site on the other. See, for example, U.S. Pat. Nos. 5,356,802; 5,436, 150 and 5,487,994; as well as Li et al. Proc., Natl. Acad. Sci. USA 89 (1992):4275-4279; Li et al. Proc. Natl. Acad. Sci. USA, 90:2764-2768 (1993); Kim et al. Proc. Natl. Acad. Sci. USA. 91:883-887 (1994a); Kim et al. J. Biol. Chem. 269:31,978-31,982 (1994b). One or more of these enzymes (or enzymatically functional fragments thereof) can be used as a source of cleavage domains.

Exemplary Type IIS restriction enzymes are described in International Publication WO 07/014275. Additional restriction enzymes also contain separable binding and cleavage domains. See, for example, Roberts et al. Nucleic Acids Res., 31:418-420 (2003). In certain embodiments, the cleavage domain includes one or more engineered cleavage half-domain (also referred to as dimerization domain mutants) that minimize or prevent homodimerization, as described, for example, in U.S. Published Application Nos. 2005/0064474, 2006/0188987, and 2008/0131962. In certain embodiments the cleavage half domain is a mutant of the wild type Fok I cleavage half domain. In some embodiments the cleavage half domain is a wild type Fok I mutant where one or more amino acid residues at positions 446, 447, 479, 483, 484, 486, 487, 490, 491, 496, 498, 499, 500, 531, 534, 537, and 538 is substituted. See, e.g., Example 1 of WO 07/139898, with amino acid residues in the Fok I protein numbered according to Wah et al, (1998) Proc. Natl. Acad. Sci. USA 95: 10564-10569. In some embodiments the cleavage half domains are modified to include nuclear or other localization signals, peptide tags, or other binding domains.

The DNA-binding domain, which can, in principle, be designed to target any genomic location of interest, can be a tandem array of Cys₂His₂ zinc fingers, each of which generally recognizes three to four nucleotides in the target DNA sequence. The Cys₂His₂ domain has a general structure: Phe (sometimes Tyr)-Cys-(2 to 4 amino acids)-Cys-(3 amino acids)-Phe(sometimes Tyr)-(5 amino acids)-Leu-(2 amino acids)-His-(3 amino acids)-His. By linking together multiple fingers (the number varies: three to six fingers have been used per monomer in published studies), ZFN pairs can be designed to bind to genomic sequences 18-36 nucleotides long.

Another type of zinc finger that binds zinc between 2 pairs of cysteines has been found in a range of DNA binding proteins. The general structure of this type of zinc finger is: Cys-(2 amino acids)-Cys-(13 amino acids)-Cys-(2 amino acids)-Cys. This is called a Cys₂Cys₂ zinc finger. It is found in a group of proteins known as the steroid receptor superfamily, each of which has 2 Cys₂Cys₂ zinc fingers.

The DNA-binding domain of a ZFN can be composed of two to six zinc fingers. Each zinc finger motif is typically considered to recognize and bind to a three-base pair sequence and as such, a protein including more zinc fingers targets a longer sequence and therefore may have a greater specificity and affinity to the target site. Zinc finger binding domains can be “engineered” to bind to a predetermined nucleotide sequence. See, for example, Beerli et al. Nature Biotechnol. 20: 135-141 (2002); Pabo et al. Ann. Rev. Biochem. 70:313-340 (2001); Isalan et al., Nature Biotechnol. 19:656-660 (2001); Segal et al. Curr. Opin. Biotechnol. 12:632-637 (2001); Choo et al., Curr. Opin. Struct. Biol. 10:41 1-416 (2000). Consequently, zinc finger binding domains can be engineered to have a different binding specificity, compared to a naturally-occurring zinc finger protein.

Standard ZFNs fuse the cleavage domain to the C-terminus of each zinc finger domain. In order to allow the two cleavage domains to dimerize and cleave DNA, the two individual ZFNs must bind opposite strands of DNA with their C-termini a certain distance apart. As discussed above, the most commonly used linker sequences between the zinc finger domain and the cleavage domain requires the 5′ edge of each binding site to be separated by 5 to 7 bp.

Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage. In certain embodiments fusion proteins target a single-stranded cleavage in a double-stranded section of DNA. Fusion proteins of this type are sometimes referred to as nickases, and can in some embodiments be preferred to limit undesired mutations. In some cases a nickase is created by blocking or limiting the activity of one half of a fusion half-domain dimer.

Engineering methods include, but are not limited to, rational design and various types of empirical selection methods. Rational design includes, for example, using databases including triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6, 140,081; 6,453,242; 6,534,261; 6,610,512; 6,746,838; 6,866,997; 7,067,617; U.S. Published Application Nos. 2002/0165356; 2004/0197892; 2007/0154989; 2007/0213269; and International Patent Application Publication Nos. WO 98/53059 and WO 2003/016496.

### iii. Transcription Activator-Like Effector Nucleases

In some embodiments, the element that induces a single or a double strand break in the target cell's genome is a nucleic acid construct or constructs encoding a transcription activator-like effector nuclease (TALEN). TALENs have an overall architecture similar to that of ZFNs, with the main difference that the DNA-binding domain comes from TAL effector proteins, transcription factors from plant pathogenic bacteria. The DNA-binding domain of a TALEN is a tandem array of amino acid repeats, each about 34 residues long. The repeats are very similar to each other; typically they differ principally at two positions (amino acids 12 and 13, called the repeat variable diresidue, or RVD). Each RVD specifies preferential binding to one of the four possible nucleotides, meaning that each TALEN repeat binds to a single base pair, though the NN RVD is known to bind adenines in addition to guanine. TAL effector DNA binding is mechanistically less well understood than that of zinc-finger proteins, but their seemingly simpler code could prove very beneficial for engineered-nuclease design. TALENs also cleave as dimers, have relatively long target sequences (the shortest reported so far binds 13 nucleotides per monomer) and appear to have less stringent requirements than ZFNs for the length of the spacer between binding sites. Monomeric and dimeric TALENs can include more than 10, more than 14, more than 20, or more than 24 repeats.

Methods of engineering TAL to bind to specific nucleic acids are described in Cermak, et al, Nucl. Acids Res. 1-11 (2011). US Published Application No. 2011/0145940, which discloses TAL effectors and methods of using them to modify DNA. Miller et al. Nature Biotechnol 29: 143 (2011) reported making TALENs for site-specific nuclease architecture by linking TAL truncation variants to the catalytic domain of Fok I nuclease. The resulting TALENs were shown to induce gene modification in immortalized human cells. General design principles for TALE binding domains can be found in, for example, WO 2011/072246.

TALENs using the +63 C-terminal truncation have been shown to cleave over a wide range of spacers. This makes design of TALENs easier and increases the number of potential sequences that can be targeted, but it also increases the number of potential regions of the genome that could be cleaved through off-target activity.

There are numerous strategies for creating the fusion proteins described above. These will typically involve joining the DNA binding domain to the cleavage domain or half domain by an operable linker. For instance in typical ZFN with a Fok I cleavage domain cleavage is obtained when the zinc finger proteins bind to target sites separated by approximately 5-6 base pairs. A linker, typically a flexible linker rich in glycine and serine, is used to join each zinc finger binding domain to the cleavage domain See, e.g., U.S. Published Application No. 2005/0064474 and PCT Application WO 07/139898. In some embodiments the engineered nuclease may use modified linkers, linkers that are longer or shorter, more or less rigid, etc. than those conventionally employed for created ZFN or TALEN fusion proteins. The linker may form a stable alpha helix linker. See, e.g., Yan et al. Biochemistry, 46:8517-24 (2007) and Merutka and Stellwagen, Biochemistry, 30:4245-8 (1991). Although the methods described herein are flexible to produce nucleases having a range of linkers, in some embodiments the linkers will be preferentially less than 50 base pairs, less than 30 base pairs, less than 20 base pairs, less than 15 base pairs, or less than 10 base pairs in length.

### b. Gene Altering Polynucleotides

The nuclease activity of the genome editing systems described herein cleave target DNA to produce single or double strand breaks in the target DNA. Double strand breaks can be repaired by the cell in one of two ways: non-homologous end joining, and homology-directed repair. In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence is used as a template for repair of the cleaved target DNA sequence, resulting in the transfer of genetic information from a donor polynucleotide to the target DNA. As such, new nucleic acid material can be inserted/copied into the site.

Therefore, in some embodiments, the genome editing composition optionally includes a donor polynucleotide. The modifications of the target DNA due to NHEJ and/or homology-directed repair can be used to induce gene correction, gene replacement, gene tagging, transgene insertion, nucleotide deletion, gene disruption, gene mutation, etc.

Accordingly, cleavage of DNA by the genome editing composition can be used to delete nucleic acid material from a target DNA sequence (e.g., to disrupt a gene that makes cells susceptible to infection (e.g., the CCRS or CXCR4 gene, which makes T cells susceptible to HIV infection), to remove disease-causing trinucleotide repeat sequences in neurons, to create gene knockouts and mutations as disease models in research, etc.) by cleaving the target DNA sequence and allowing the cell to repair the sequence in the absence of an exogenously provided donor polynucleotide. Thus, the subject methods can be used to knock out a gene (resulting in complete lack of transcription or altered transcription) or to knock in genetic material into a locus of choice in the target DNA.

Alternatively, if the genome editing composition includes a donor polynucleotide sequence that includes at least a segment with homology to the target DNA sequence, the methods can be used to add, i.e., insert or replace, nucleic acid material to a target DNA sequence (e.g., to “knock in” a nucleic acid that encodes for a protein, an siRNA, an miRNA, etc.), to add a tag (e.g., 6× His, a fluorescent protein (e.g., a green fluorescent protein; a yellow fluorescent protein, etc.), hemagglutinin (HA), FLAG, etc.), to add a regulatory sequence to a gene (e.g., promoter, polyadenylation signal, internal ribosome entry sequence (IRES), 2A peptide, start codon, stop codon, splice signal, localization signal, etc.), to modify a nucleic acid sequence (e.g., introduce a mutation), and the like. As such, the compositions can be used to modify DNA in a site-specific, i.e., “targeted”, way, for example gene knock-out, gene knock-in, gene editing, gene tagging, etc. as used in, for example, gene therapy, e.g., to treat a disease or as an antiviral, antipathogenic, or anticancer therapeutic.

In applications in which it is desirable to insert a polynucleotide sequence into a target DNA sequence, a polynucleotide including a donor sequence to be inserted is also provided to the cell. By a “donor sequence” or “donor polynucleotide” or “donor oligonucleotide” it is meant a nucleic acid sequence to be inserted at the cleavage site. The donor polynucleotide typically contains sufficient homology to a genomic sequence at the cleavage site, e.g., 70%, 80%, 85%, 90%, 95%, or 100% homology with the nucleotide sequences flanking the cleavage site, e.g., within about 50 bases or less of the cleavage site, e.g., within about 30 bases, within about 15 bases, within about 10 bases, within about 5 bases, or immediately flanking the cleavage site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) will support homology-directed repair. Donor sequences can be of any length, e.g., 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence includes a non-homologous sequence flanked by two regions of homology, such that homology-directed repair between the target DNA region and the two flanking sequences results in insertion of the non-homologous sequence at the target region.

Donor sequences can also include a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence can include certain sequence differences as compared to the genomic sequence, e.g., restriction sites, nucleotide polymorphisms, selectable markers (e.g., drug resistance genes, fluorescent proteins, enzymes etc.), etc., which can be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor sequence can be a single-stranded DNA, single-stranded RNA, double-stranded DNA, or double-stranded RNA. It can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. Proc. Natl. Acad. Sci. USA 84:4959-4963 (1987); Nehls et al. Science 272:886-889 (1996). Additional methods for protecting exogenous polynucleotides from degradation include, but are not limited to, addition of terminal amino group(s) and the use of modified internucleotide linkages such as, for example, phosphorothioates, phosphor amidates, and O-methyl ribose or deoxyribose residues.

As an alternative to protecting the termini of a linear donor sequence, additional lengths of sequence can be included outside of the regions of homology that can be degraded without impacting recombination. A donor sequence can be introduced into a cell as part of a vector molecule having additional sequences such as, for example, replication origins, promoters and genes encoding antibiotic resistance.

### 2. Injection Formulations

The injection formulations disclosed herein typically include an effective amount of a genome editing composition in a pharmaceutically acceptable carrier suitable for hydrodynamic injection.

### a. Pharmaceutical Compositions

The formulations can include a physiologically-acceptable carrier (such as physiological saline or phosphate buffer) selected in accordance with the route of administration and standard pharmaceutical practice. Pharmaceutical compositions including the genome editing compositions are prepared according to standard techniques and include a pharmaceutically acceptable carrier. In some embodiments, normal saline is employed as the pharmaceutically acceptable carrier. Other suitable carriers include, e.g., water, buffered water, 0.9% saline, 0.3% glycine, and the like, including glycoproteins for enhanced stability, such as albumin, lipoprotein, globulin, etc. The resulting pharmaceutical preparations can be sterilized by conventional, well known sterilization techniques. The aqueous solutions can then be packaged for use or filtered under aseptic conditions and lyophilized, the lyophilized preparation being combined with a sterile aqueous solution prior to administration. The compositions cab contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc.

In a preferred embodiments, the formulation is a 0.9% sodium chloride solution.

The genome editing compositions in the pharmaceutical formulations can vary widely, i.e., from less than about 0.01%, usually at or at least about 0.05-5% to as much as 10 to 30% by weight and will be selected primarily by fluid-volumes, viscosities, etc., in accordance with the particular mode of administration selected, as well as the desired total volume and injection speed as discussed in more detail below. For example, the concentration can be increased to lower the fluid load associated with treatment. This can be particularly desirable in patients having atherosclerosis-associated congestive heart failure or severe hypertension. Alternatively, the compositions can be diluted to low concentrations to lessen-inflammation at the site of administration.

The pharmaceutical compositions can be formulated as a pharmaceutical dosage unit, also referred to as a unit dosage form that contains an effective amount (e.g., dosage) of a genome editing composition.

### b. Delivery Vehicles

The compositions can be administered and taken up into the cells of a subject with or without the aid of a delivery vehicle. For example, nucleic acids may also be delivered by other carriers, including liposomes, polymeric micro- and nanoparticles and polycations such as asialoglycoprotein/polylysine, which can enhance transfection efficiency. In some embodiments, the composition is incorporated into or encapsulated by a nanoparticle, microparticle, micelle, synthetic lipoprotein particle, or carbon nanotube. Preferred carriers include targeted liposomes (Liu, et al. Curr. Med. Chem., 10:1307-1315 (2003)) such as immunoliposomes, which can incorporate acylated mAbs into the lipid bilayer. Polycations such as asialoglycoprotein/polylysine may be used, where the conjugate includes a molecule which recognizes the target tissue (e.g., asialoorosomucoid for liver) and a DNA binding compound to bind to the DNA to be transfected. Polylysine is an example of a DNA binding molecule which binds DNA without damaging it. This conjugate is then complexed with plasmid DNA for transfer.

Additional considerations for nucleic acid delivery in vivo are discussed in Kanasty, et al., Nat Mater 12, 967-977 (2013), which is specifically incorporated by reference herein in its entirety, and can be used to further modify the formulations disclosed herein.

### c. Effective Amounts and Dosages

Typically the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

Typically, the formulations include an amount of genome editing composition effective to modify the genome of one or more targets cells in a subject following hydrodynamic administration of the formulation to the subject. In preferred embodiments, the amount is effective to modify the genome of enough of the target cells to treat, reduce, or prevent one or symptoms a disease being treated, or to produce an alteration in a physiological or biochemical manifestation thereof.

As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration (e.g., systemic circulation, regional or local circulation, etc.), and on the duration of the treatment desired.

As the genome editing composition is typically, one or more nucleic acid constructions, the dosages described herein are typically nucleic acid dosages. The Examples below disclose administration of 60 μg plasmid DNA (encoding sgRNA and Cas9) and 60 μg ssDNA oligo (donor polynucleotide) by tail vein injection (systemic circulation) in rodents. Khorsandi, et al., Cancer Gene Therapy, 15:225-230 (2008) reported administering pigs dosages of between 10 mg and 20 mg of plasmid, and humans dosages of 1 mg to 45 mg of plasmid by selective hydrodynamic injection (regional circulation), and all dosages were found to be safe and tolerated by the subjects. Therefore, generally, the dosages can range from about 0.001 mg to about 1,000 mg, more preferable about 0.01 mg to about 100 mg of each component of the genome editing composition (e.g., each plasmid, donor polynucleotide, etc.), depending on the subject to be treated, the route of administration, the targets cells, etc. It will be appreciated that generally large animals (e.g., primates) may require a larger dosage than small animals (e.g., rodents) and systemic circulation may require a larger dosage than regionally or locally restricted administration.

### B. Methods of Delivery

The Examples below illustrate that genome editing can be carried out in an adult mammal in an effective amount to treat, and in some cases, cure genetic diseases. In the Examples below, the compositions for genome editing are delivered using hydrodynamic injection. Therefore, in preferred embodiments, genome editing compositions are administered to a subject using hydrodynamic injection.

Hydrodynamic injection, also referred to as high pressure injection, is a method of administering nucleic acids in vivo. Hydrodynamic injection is amenable to delivery of “naked” nucleic acids, and therefore does not require viral carriers that can require laborious procedures for preparation and purification, and carry with them concerns about the possibility for recombination with endogenous virus to produce a deleteriously infectious form. Hydrodynamic injection also does not appear to cause the immune response and other side effects that render the repeated administration of viral vectors problematic. Being different from carrier-based strategy and the earlier work employing hypertonic solution and elevated hydrostatic pressure to facilitate intracellular DNA transfer, hydrodynamic gene delivery relies on hydrodynamic pressure generated by a rapid injection of a large volume of fluid into a blood vessel to deliver genetic materials into parenchyma cells.

The fundamental mechanisms underlying hydrodynamic gene delivery are described in Suda and Liu, et al., Molecular Therapy, 15(12):2063-2069 (2007), Al-Dosari, et al., Adv. Genet. 54: 65-82 (2005), Kobayashi, et al., Adv Drug Deliv. Rev. 57:713-731 (2005), and Herweijer and Wolff, Gene Ther. 14: 99-107 (2007). Briefly, hydrodynamic gene delivery was developed based on the structure and properties of blood capillaries, and the dynamic properties of fluids passing through blood vasculature. Parenchyma cells are the primary target because capillary endothelium and parenchyma cells are closely associated, allowing for immediate access of DNA to parenchyma cells once the endothelial barrier is disrupted. In addition, the capillary wall is thin, stretchable and relatively easy to break. Hydrodynamic gene delivery uses a hydrodynamic force generated by a pressurized injection of a large volume of DNA solution into the blood vessel so as to permeabilize the capillary endothelium and generate “pores” in the plasma membrane of the surrounding parenchyma cells, through which DNA or other macromolecules of interest can reach the cell interior. Subsequently, the membrane pores close, trapping these molecules inside. The overall gene delivery efficiency of the hydrodynamic procedure is determined by the capillary structure, architecture of cells surrounding the capillary, and the hydrodynamic force applied to the interior of the vasculature.

Therefore, the polynucleotide(s) of a genome editing composition can be delivered into parenchymal cells by administering the composition into a vessel under conditions that increase the pressure against vessel walls and thereby increase the permeability of the vessel. Typically, the increase in pressure is driven by increasing the volume of fluid within the vessel. Therefore, a genome editing composition is typically delivered into the mammalian vessel in a volume of solution and over a time period effective to increase the pressure against vessel walls and thereby increase the permeability of the vessel enough for the polynucleotide(s) to be transfected into the parenchymal cells. The pressure, and therefore the permeability of the blood vessels and associated access to the parenchymal cells can be controlled by altering the specific volume of the solution in relation to the specific time period of administration as discussed in more detail below.

### 1. Systemic Circulation

In some embodiments, the method includes hydrodynamic delivery of a genome editing composition via systemic circulation. Methods and guidelines for systemic administration of plasmid DNA by hydrodynamic injection are discussed in Liu, et al., Gene Therapy, 6:1258-1266 (1999), and elsewhere. Typically, systemic hydrodynamic administration includes quickly injecting into a blood vessel of a subject a large volume of liquid carrier under high pressure. In the case of rodents, a preferred blood vessel is the tail vein. It is believed that upon rapidly injecting a solution into the tail vein, the fluids enter the vena cava where the fluids back up because the large volume cannot be pumped rapidly enough through the heart. This creates increased pressure in the vena cava and pushes the DNA solution into draining vasculature, in particular, the large hepatic vein. There, the fluid is forced out of the capillaries into the tissue and the nucleic acids (or other compounds present in the solution) enter into the parenchymal cells. Hydrodynamic tail vein delivery mainly results in expression in the liver, however, levels of expression are found in the spleen, heart, kidneys and lungs.

Therefore in some embodiments, the genome editing composition is delivered systemically or by systemic circulation using hydrodynamic injection. Although this method is particularly safe and effective for delivery of nucleic acids in rodents, studies show that the heavy overload of fluid in the systemic circulation can induce irregularity in cardiac function and lead to transient heart failure. These results have raised concerns that cardiac congestion, although well tolerated by rodents, may not be safe for routine use in human subjects. It is noted that the Examples below show that in some cases, a single systemic administration of the CRISPR/Cas-mediated genome editing composition induced a permanent genetic alteration in an effective amount of cells to reduce the phenotype associated with a genetic disease in the liver. Therefore, it is believed that in some embodiments, the risks of systemic circulation using hydrodynamic injection in humans will be tolerable, particularly when a single administration is effective to reduce or prevent one of more symptoms of the disease being treated.

Systemic circulation can be achieved in humans using other blood vessels where the same or similar hydrodynamic results are achieved using the tail vein in rodents. For example, a similar distribution of transfection and expression has been observed after delivery into the jugular vein of mice and chickens (Hen, et al., Domest. Anim. Endocrinol., 30:135-143 (2005)). Accordingly, in some embodiments, systemic circulation is achieved by administration through the jugular vein. Other suitable points of entry are known in the art and discussed in more detail below.

### 2. Regional and Local Circulation

In some embodiments, the method includes hydrodynamic delivery of a genome editing composition via regional or local circulation. Methods and guidelines for regional administration of plasmid DNA by hydrodynamic injection are discussed in, for example, Suda and Liu, et al., Molecular Therapy, 15(12):2063-2069 (2007), Al-Dosari, et al., Adv. Genet. 54: 65-82 (2005), Kobayashi, et al., Adv. Drug Deliv. Rev. 57: 713-731 (2005), Herweijer and Wolff, Gene Ther. 14: 99-107 (2007), Hagstrom, et al., Molecular Therapy, 10(2):386-398 (2004); Lewis and Wolff, Advanced Drug Delivery Reviews 59:115-123 (2007), U.S. Published Application Nos. 2004/0259828 and 2007/0244067, U.S. Pat. Nos. 8,021,686, 7,803,782, 7,781,415, 7,642,248, 7,547,683, 7,396,821, 7,148,205, 7,144,869, 7,098,030, 6,867,196, 6,379,966, and 6,627,616.

Regional and local hydrodynamic administration includes quickly injecting into a blood vessel of a subject a large volume of liquid carrier under high pressure, in such a way that the injected solution is forced into an organ, tissue, or cells of interest as discussed above for systemic circulation, but without flooding the vena cava or causing the heart related complications discussed above for systemic circulation. Typically, this is accomplished by directing the injected solution and associated pressure in a particular direction of interest (e.g., the target organ, tissue, or cells and away from the heart and/or other non-target organs, tissues, or cells).

Directing the injected solution can be accomplished by reducing or preventing the flow of the solution from traveling down the vessel in at least one direction distal to the target organ, tissue, or cells. For example, in some embodiments, the solution is forced in the direction of interest (e.g., proximal to the target organ, tissue, or cells) by reducing or preventing back flow. Therefore, in some embodiments, the solution is forced preferentially in the direction of injection and reduced or prevented from flowing against the direction of injection.

By delivering the solution directly into vessels supplying an organ, it is possible to direct a majority of the expression to that organ. For example, Maruyama et al., demonstrated that delivery into the renal vein resulted in high reporter gene expression levels in the kidneys (Maruyama, et al., Mol Biotechnol., 27: 23-31 (2004) and Kameda, Biochem. Biophys. Res Commun., 314: 390-395 (2004). Hydrodynamic injection into the common carotid artery led to transfection of 10-30% of the cells in brain tumors implanted in the frontal lobe of rats and skeletal limb muscle tissues were transfected following tail vein delivery, when occluding the aorta and vena cava, thus forcing the pDNA solution into the hindlimbs (Barnett, et al., Gene Therapy, 11:1283-1289 (2004) and Liang, et al., Gene Therapy, 11:901-908 (2004)). Therefore, in some embodiments the solution is delivered into a vessel that supplies the organ, tissue, or cells of interest. In further embodiments, the vessel is occluded on side of the site which is distal to the organ, tissue or cells of interest. Other vessels that are not the vessel to which the solution is delivered can also be occluded in manner that further increases concentration of volume and associate pressure from the solution in the target organ, tissue, or cells.

In particular embodiments, the delivery method is hydrodynamic limb vein (HLV) or hydrodynamic limb artery (HLA) delivery. In HLV, the solution is rapidly delivered anterograde into a limb vein while the blood flow in and out of the limb is reduced by a tourniquet (Hagstrom, et al., Mol Ther, 10:386-398 (2004)). The tourniquet placement allows a transient increase in vascular pressure upon injection. DNA is only extravasated in areas of increased pressure, thus limiting gene transfer to the isolated limb. It has been reported that a complete procedure can be accomplished transcutaneously in 5-10 min. The method can be optimized by varying known catheterization and tourniquet placement techniques and delivery parameters (injection volume and rate) (Herweijer and Wolff, Gene Ther. 14: 99-107 (2007)). This technique is simple (especially in larger animals such as humans) and results in high gene transfer efficiency to skeletal muscle cells (10-40%). HLA is an analogous procedure where the solution is delivered into an artery instead of a vein.

### 3. Parameters of Hydrodynamic Delivery

As discussed above, a variety of parameters including solution volume, delivery rate, and the type or location of vessel occlusion can be manipulated to fine tune the efficiency of delivery of the gene editing polynucleotides into the target cells. The specific parameters for achieving optimal increases in vascular permeability in test subjects such as laboratory animals as well as human subjects are well within the level of skill in the arts of animal science, anatomy, physiology, pharmacology and clinical medicine.

### a. Injection Volume

Optimal injection volume is related to the size of the animal to be injected as well as target tissue volume or surface area. For example, Liu, et al., Gene Therapy, 6:1258-1266 (1999) reported that optimal gene expression in mice was obtained by systemic circulation at approximately 1.2, 1.6 and 3.0 ml for animals with body weights of 11-13, 18-20, and 30-32 g, respectively, indicating that optimal transgene expression can be obtained using an injection volume (e.g., ml) for systemic circulation that is approximately 8-12% of the body weight (e.g., grams) of the animal. This correlation is likely related to the blood volume and cardiac capacity of the animal, therefore, since younger animals have a smaller cardiac output, a smaller injection volume can achieve the desired hydrostatic pressure for high level gene expression. In some embodiments, the volume roughly equates to the total blood volume (e.g., 7.3% of body weight in mice). In some embodiments, volumes in terms of ml/body weight can be 0.01 ml/g to 0.1 ml/g, or 0.03 ml/g to 0.1 ml/g, or greater. Elsewhere, injection volumes of 70 to 200 ml have also been reported for primates (U.S. Published Application No. 2002/0132788).

Studies indicate that acceptable levels of gene expression can achieved by regional or circulation at substantially lower volumes. For example, Khorsandi, et al., Cancer Gene Therapy, 15:225-230 (2008) tested volumes of 300 ml in pigs and humans using a minimally invasive method of hydrodynamic delivery to selective liver segments using balloon catheters. Likewise, Zhang et al. have shown in rats that the volume required for successful hydrodynamic gene delivery can be reduced to about 3 ml in a 200-250 g rat (<1.5% of body weight) by targeting an isolated liver through the hepatic vein, Eastman et al. demonstrated in rabbits that a volume of 15 ml/kg was sufficient for successful gene transfer into liver cells, Yoshino et al. and Alino et al. showed that hydrodynamic injection of 100-150 ml of DNA solution into the hepatic or portal vein of pigs is safe 26, 28 39,53. Therefore, volumes of 50 ml-1,000 ml, preferably 100 ml-750 ml, more preferably, 300 ml-750 ml, most preferably, 500-750 ml, can be administered to an average adult human, for example a 50 kg human. Volumes of >5 ml per rat limb or >70 ml for a primate have been reported for regional delivery to skeletal muscle, with concomitant external application of pressure e.g., with a cuff or tourniquet, such that pressure within the vessel is increased and permeability to outward movement of polynucleotide, etc. is enhanced.

### b. Injection Speed

Injection speed can also be important consideration in the efficacy of hydrodynamic delivery, and dependent on the subject animal, the size of the vessel to be injected into, and the type of administration (e.g., systemic circulation, regional or local circulations, etc.). Generally, the combination of solution volume and rate of administration should be effective to permeabilize the capillary endothelium and allow the polynucleotides within to contact the underlying parenchymal. More preferably the combination of solution volume and rate of administration is effective to generate “pores” in the plasma membrane of the target parenchyma cells, through which DNA or other macromolecules of interest can reach the cell interior.

In mice, reducing the injection speed from 5 seconds to 30 seconds for a 1.6 ml volume caused a 4,500 fold decrease in gene expression. Therefore, when a range of speeds are provided, the faster injection speeds are generally preferred to the extent they are safe and tolerated in the subject. Khorsandi, et al., Cancer Gene Therapy, 15:225-230 (2008) reports safely and effectively administering to pigs volumes of 200-300 ml of solution at a rates of 2 ml/s to 20 ml/s and injection pressures of 750 psi to 1,100 psi. Others report a total injection volume (6-35 mls) can be injected into the vascular system of rats from 20 to 7 seconds, and a total injection volume of 80-200 mls can be injected into the vascular system of monkeys in 120 seconds or less. U.S. Pat. No. 7,642,248, reports injection rates of less than 0.012 ml per gram (animal weight) per second can be used for large injection volumes, while injection rates of less than ml per gram (target tissue weight) per second can be used for gene delivery to target organs, and injection rates of less than 0.06 ml per gram (target tissue weight) per second are used for gene delivery into limb muscle and other muscles of primates.

The solution can be delivered by any means suitable for delivering the desired volume at the desired rate. For example, the solution can be administered using an injection device such as a catheter, syringe needle, cannula, stylet, balloon catheter, multiple balloon catheter, single lumen catheter, and multilumen catheter. Single and multi-port injectors may be used, as well as single or multi-balloon catheters and single and multilumen injection devices. A catheter can be inserted at a distant site and threaded through the lumen of a vein so that it resides in or near a target tissue. The injection can also be performed using a needle that traverses the skin and enters the lumen of a vessel.

Administration can be aided by the incorporation of pump or other system to facilitate delivery of the desired volume at the desired pressure. In a particular embodiment, administration includes use of a computer-assisted system enabling real-time control of the injection based on the hydrodynamic pressure at the injection site of the tissue. Precise control of injection can avoid tissue damage caused by too heavy an injection, or low gene delivery efficiency due to insufficient volume or injection speed.

Gene delivery can also be optimized and toxicity (tissue damage) minimized by varying the volume of the solution and the speed of injection; varying the osmotic pressure by the addition of mannitol to the injection solution; increasing fluid and DNA extravasation, e.g., by vessel dilation using papaverine, hyaluronidase, or VEGF protein pre-injection.

### c. Occluding Vessels

In some embodiments one or more vessels are occluded to reduce or prevent flow of the solution in one or more directions, for example, back flow. Methods of occluding vessels are known in the art and can be accomplished in a variety of manners. For example, in some embodiments, the injection apparatus itself reduces back flow. In some embodiments one or more cuffs, tourniquets or combination thereof is used to reduce or prevent solution flow in one or more directions. The cuff or tourniquets can be applied directly to the vessel, or to the tissue surrounding the vessel. In some embodiments, one or more balloon catheters is used to reduce or prevent solution flow in one or more directions.

The occlusion(s) can be carried out using non-invasive procedures, minimally invasive procedures, or invasive procedures. For example, in some embodiment, the vessel or vessels are occluded by an open surgical procedure. In other embodiments, the vessel or vessels are occluded using a minimally invasive procedure such as percutaneous surgery. Accordingly, various approaches can be carried out through the skin or through a body cavity or anatomical opening and may incorporate the use of catheters, arthroscopic devices, laparoscopic devices, and the like, and remote-control manipulation of instruments with indirect observation of the surgical field through an endoscope or large scale display panel, etc. Non-invasive methods can include, for example, external application of a cuff, wrap, or tourniquet to the subject in a way that reduces flow of the solution away from the target organ, tissue, or cells.

Occlusion of vessels including, but not limited to balloon catheters, clamps, tourniquets or cuffs can limit or define the target area. Preferably, the processes require that blood flow be impeded is substantially less time than is required to cause tissue damage by ischemia.

One method for occluding fluid flow is the application of an external cuff. The term cuff means an externally applied device for impeding fluid flow to and from a mammalian limb. The cuff applies compression around the limb such that vessels, in an area underneath the cuff, are forced to occlude an amount sufficient to impede fluid from flowing through the vessels at a normal rate. One example of a cuff is a sphygmomanometer, which is normally used to measure blood pressure. Another example is a tourniquet. A third example is a modified sphygmomanometer cuff containing two air bladders such as is used for intravenous regional anesthesia (i.e., Bier Block). Double tourniquet, double cuff tourniquet, oscillotonometer, oscillometer, and haemotonometer are also examples of cuffs. A sphygmamanometer can be inflated to a pressure above the systolic blood pressure, above 500 mm Hg or above 700 mm Hg or greater than the intravascular pressure generated by the injection.

### 4. Sites of Administration

The disclosed hydrodynamic delivery methods are typically intravascular delivery methods. Intravascular refers to a route of administration that enables a gene editing composition to be delivered to cells more evenly distributed than direct injections. Intravascular refers an internal tubular structure, also referred to herein as a vessel that is connected to a tissue or organ within the body of an animal such as a mammal Within the cavity of the tubular structure, a bodily fluid flows to or from the body part. Examples of bodily fluid include blood, lymphatic fluid, or bile. Examples of vessels include arteries, arterioles, capillaries, venules, sinusoids, veins, lymphatics, and bile ducts. The intravascular route includes delivery through the blood vessels such as an artery or a vein.

Exemplary routes used for hydrodynamic injection include the tail vein, tail artery, inferior vena cava, superior vena cava, jugular vein, hepatic vein, hepatic artery, portal vein, bile duct, saphenous, cephalic and median veins, femoral vein, femoral artery, brachial and popliteal arteries, iliac arteries, renal vein, carotid artery, and aorta.

In particular embodiments the hydrodynamic injection is HLV. Suitable sites for HLV are typically veins present in the limb distal to the site of occlusion. A preferred vein is a superficial vein. Exemplary limb veins include the cephalic vein, median vein, median cephalic, median basilica, brachial vein, basilic vein, interosseous vein, radial vein, ulnar vein (anterior, posterior, common), deep palmar veins, great saphenous vein (medial saphenous vein, v. saphena magna, internal saphenous vein, long saphenous vein), lesser saphenous vein, small saphenous vein (lateral saphenous vein, external saphenous vein, v. saphena parca, short saphenous vein), anterior tibial vein, posterior tibial vein, peroneal vein, popliteal vein, plantar vein (medial and lateral), dorsal venous arch, dorsal digital vein, dorsal metacarpal vein and dorsal pedis vein.

### 5. Target Organs, Tissues, and Cells

The target cells, and therefore the particular method of hydrodynamic injection, are typically selected based on disease to be treated. In some embodiments, the target cells are liver cells, spleen cells, heart cells, kidney cells, lung cells, skeletal muscle cells (myofiber, myocytes) bone cells (osteocytes, osteoclasts, osteoblasts), bone marrow cells, stroma cells, joint cells (synovial and cartilage cells), connective tissue cells (fibroblasts, fibrocytes, chondrocytes, mesenchyme cells, mast cells, macrophages, histiocytes), cells in tendons, cells in the skin, or cells in the lymph nodes.

In preferred embodiments, the target cells are parenchymal cells. Parenchymal cells are the distinguishing cells of a gland or organ contained in and supported by the connective tissue framework. The parenchymal cells typically perform a function that is unique to the particular organ. The term “parenchymal” often excludes cells that are common to many organs and tissues such as fibroblasts and endothelial cells within blood vessels.

In a liver organ, the parenchymal cells include hepatocytes, Kupffer cells and the epithelial cells that line the biliary tract and bile ductules. The major constituent of the liver parenchyma are polyhedral hepatocytes (also known as hepatic cells) that presents at least one side to an hepatic sinusoid and opposed sides to a bile canaliculus. Liver cells that are not parenchymal cells include cells within the blood vessels such as the endothelial cells or fibroblast cells.

In striated muscle, the parenchymal cells include myoblasts, satellite cells, myotubules, and myofibers. In cardiac muscle, the parenchymal cells include the myocardium also known as cardiac muscle fibers or cardiac muscle cells and the cells of the impulse connecting system such as those that constitute the sinoatrial node, atrioventricular node, and atrioventricular bundle.

### III. Diseases to be Treated

Applications of the disclosed compositions and methods include gene therapy, e.g., to treat a disease, or as an antiviral, antipathogenic, or anticancer therapeutic. The disclosed methods can be used to treat any disease or condition wherein genome modification of target cells is effective to treat the disease or condition, and wherein the target cells can be transfected with the disclosed compositions by hydrodynamic injection.

Cas9 has been used to carry synthetic transcription factors (protein fragments that turn on genes.) This enabled the activation of specific human genes. The technique achieved a strong effect by targeting multiple CRISPR constructs to slightly different spots on the gene's promoter (Pennisi, Science, 341(6148): 833-836 (2013). The genes included some tied to human diseases, including those involved in muscle differentiation, cancer, inflammation and producing fetal hemoglobin.

In preferred embodiments, the compositions and methods are used to treat a genetic disease caused by a genetic mutation. In preferred embodiments, the mutation is one that be corrected by a donor polynucleotide such as those described herein. For example, in some embodiments, the disease is caused by a point mutation. Exemplary diseases include, but are not limited to, cystic fibrosis, sickle-cell anemia and Huntington's disease which are caused by single base pair mutations.

Preferably the cells leading to the disease pathology can be transfected by hydrodynamic injection. Preferred target cells include those discussed above, for example, liver cells, spleen cells, heart cells, kidney cells, lung cells, skeletal muscle cells, etc. Exemplary diseases and conditions that have been proposed to be treated by transfecting cells through hydrodynamic injection include Fabry disease, growth hormone deficiency, hemophilia, metachromatic leukodystrophy, mucopolysaccharidosis I, phenylketonuria, short chain acyl-CoA dehydrogenase deficiency, alpha-1 antitrypsin deficiency, diabetes, obesity, myocarditis, glomerulonephritis, organophosphate toxicity, xenotransplantation, hypoxic-ischemia encephalopathy, liver regeneration, and various types of cancer (Suda and Liu, et al., Molecular Therapy, 15(12):2063-2069 (2007)). Another exemplary disease that can be treated is hereditary tyrosinemia.

The Examples below illustrate that typically, not every target cell is transfected with an effective amount of a genome editing composition to induce a permanent alteration of a genome. Therefore, it will be appreciated that the disclosed compositions and methods are particularly effective for treating diseases and conditions in which genetically altering a fraction of the target cells is effective to treat the disease or condition. In some embodiments, the disease is one in which a positive selection of “repaired” cells can enhance disease treatment. In such diseases, gene repair is required in only a small number of cells to effectively treat, or in some cases cure, the disease (Azuma, Nat. Biotechnol., 25:903-910 (2007), and Aponte, et al., Proc. Natl. Acad. Sci. USA, 98, 641-645 (2001)). For example, in some embodiments, correction of the genetic defect or error in <0.01%, or about at least 0.1%, 0.2%, 0.3%, 0.4%, 0.5%, 0.6%, 0.7%, 0.8%, 0.9%, 1%, 5%, 10%, 20%, 30%, 40%, 50%, 60%, 70%, 80%, 90% or 95% of the target cells is effective to treat the disease. In other embodiments, correction of the genetic defect or error in at least 10², 10³, 10⁴, 10⁵, 10⁶, 10⁷, 10⁸, 10⁹, 10¹⁰ or more target cells is effective to treat the disease. In some embodiments, correction of the genetic defect or error in 0.1-10%, or between 0.25%-0.5%, or 1%-75%, or 10%-50%, or 20%-40% of the target cells is effective to treat the disease.

There are several diseases in which selection has been shown, including, but not limited to, Fanconi's anemia (Battaile, Blood, 94, 2151-2158 (1999)), the copper storage disorder Wilson's disease (Allen, et al., J. Gastroenterol. Hepatol., 19, 1283-1290 (2004)), many bile-acid transporter defects (De Vree, et al., Gastroenterology, 119, 1720-1730 (2000)), junctional epidermolysis bullosa (Ortiz-Urda, et al., Gene Ther., 10, 1099-1104 (2003)), and al-antitrypsin deficiency (Ding, et al., J Clin Invest, 121, 1930-1934 (2011)). It has also been estimated that the frequency of gene correction of 10⁻² would be therapeutically beneficial in patients with hemophilia A (Kay, et al., Proc. Natl. Acad. Sci. USA 96, 9973-9975 (1999)).

In some embodiments the induced genetic alteration(s) disclosed herein are heterozygous with respect to the target cell's genome. In some embodiments the induced genetic alteration(s) disclosed herein are homozygous with respect to the target cell's genome.

The Examples below illustrate a specific embodiment in which hydrodynamic injection was used to deliver CRISPR-genome editing composition in an effective amount to correct a genetic mutation in vivo mouse model of hereditary tyrosinemia type I (HTI).

### EXAMPLES

### Example 1

### CRISPR Genome Editing Rescues FAH Deficiency In Vivo in a Mouse Model

### Materials and Methods

### Construction of CRISPR Vectors

pX330 vector expressing Cas9 and sgRNA7 was digested with BbsI. Oligos for each targeting site were annealed, phosphorylated by T4 PNK, and ligated with linearized pX330 vector. The sequence for the sgRNA are as follows:

sgRNA 1:
(SEQ ID NO: 3)
ACTGGAGCAGTAATGCCTGGTGG
sgRNA 2:
(SEQ ID NO: 6)
ACGACTGGAGCAGTAATGCCTGG
sgRNA 3:
(SEQ ID NO: 9)
CCTCATGAACGACTGGAGCAGTA

### Mice and Hydrodynamic Injection

All animal study protocols were approved by the MIT Animal Care and Use Committee. Fah^{mut/mut} mice (Paulk, et al., Hepatology, 51, 1200-1208 (2010)) were kept on 10mg/L NTBC water. Mice with more than 20% weight loss were humanely euthanized according to MIT guidelines. Vectors for hydrodynamic tail vein injection were prepared using the EndoFreeMaxi Kit (Qiagen). 199nt ssDNA ultramer oligo was from IDT. GCTTTCTTCGTAGGCCCTGGGAACAGATTCGGAGAGCCAATCCCC ATTTCCAAAGCCCATGAACACATTTTCGGGATGGTCCTCATGAAC GACTGGAGCGGTAATGCCTGGTGGCCCAGCTTCCTCTGATGTTCT GTTCTTAGGGGCACACACAGGAGTTGGGTATGGGACAGGAGGCC TAAGTACTACAGGGGTGATA (SEQ ID NO:12). The G nucleotide to correct the A→G mutation is underlined.

For hydrodynamic liver injection, plasmid DNA (60 mg) and ssDNA oligo (60 μg) suspended in 2 ml saline were injected via the tail vein in 5-7 seconds into 8-10 weeks old Fah^{mut/mut} mice. 8 weeks old female FVB mice from Jackson lab were injected with 60 μg plasmid DNA and monitored for body weight.

### Results

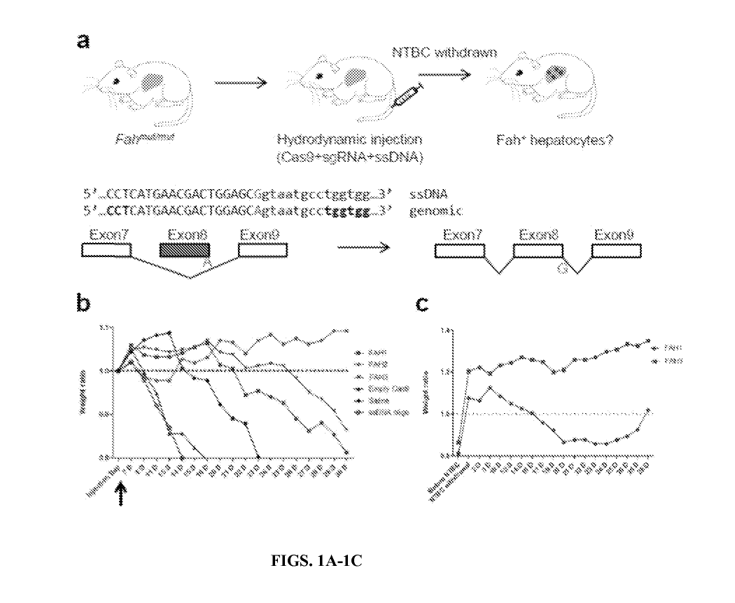

Experiments were designed to investigate the potential of CRISPR-mediated in vivo genome editing in adult animals. A mouse model of hereditary tyrosinemia type I (HTI) was utilized. HTI is a fatal genetic disease caused by mutation of fumarylacetoacetate hydrolase (FAH), the last enzyme in the tyrosine catabolic pathway (FIGS. 1A and 2A) (Paulk, et al., Hepatology, 51, 1200-1208 (2010), Azuma, et al., Nat Biotechnol., 25, 903-910 (2007)). The Fah5981SB mouse model (Paulk, et al., Hepatology, 51, 1200-1208 (2010), Aponte, et al., Proc. Natl. Acad. Sci. USA, 98, 641-645 (2001)) (termed Fah^{mut/mut} thereafter) of HTI harbors the same homozygous G→A point mutation in the last nucleotide of exon 8 as the human disease, causing splicing skipping of exon 8 and formation of truncated, unstable FAH protein. FAH deficiency causes accumulation of toxic metabolites, such as fumarylacetoacetate in hepatocytes, resulting in severe liver damage (Paulk, et al., Hepatology, 51, 1200-1208 (2010)). 2-(2-nitro-4-trifluoromethylbenzoyl)-1,3-cyclohexanedione (NTBC), a pharmacological inhibitor of the pathway upstream of FAH, rescues the phenotype and prevents acute liver injury.

A previous study showed that targeted adeno associated virus (AAV) integration by homologous recombination could achieve stable gene repair in vivo, but required multiple rounds of NTBC withdrawal and recovery (Paulk, et al., Hepatology, 51, 1200-1208 (2010)). It has been reported that liver cells genetically repaired for the Fah enzyme have a selective advantage and can expand and repopulate the liver. This liver disease and several other diseases with such positive selection are unique because gene repair is required in only a small number of cells (Azuma, et al., Nat Biotechnol., 25, 903-910 (2007), Aponte, et al., Proc. Natl. Acad. Sci. USA, 98, 641-645 (2001)). Gene repair frequency range of 1/10,000 hepatocytes was reported to rescue the phenotype of Fah^{mut/mut} mice.

In order to edit the endogenous Fah locus, three sgRNA targeting Fah were cloned into the pX330 vector (Hsu, et al., Nat Biotechnol., 31, 827-832 (2013)) co-expressing sgRNA and Cas9 (FIG. 2B-2D). To facilitate homologous recombination and correct the G→A splicing mutation, a 199nt ssDNA donor was synthesized harboring the wild-type “G” nucleotide and homologous arms flanking the sgRNA target region (FIG. 1A).

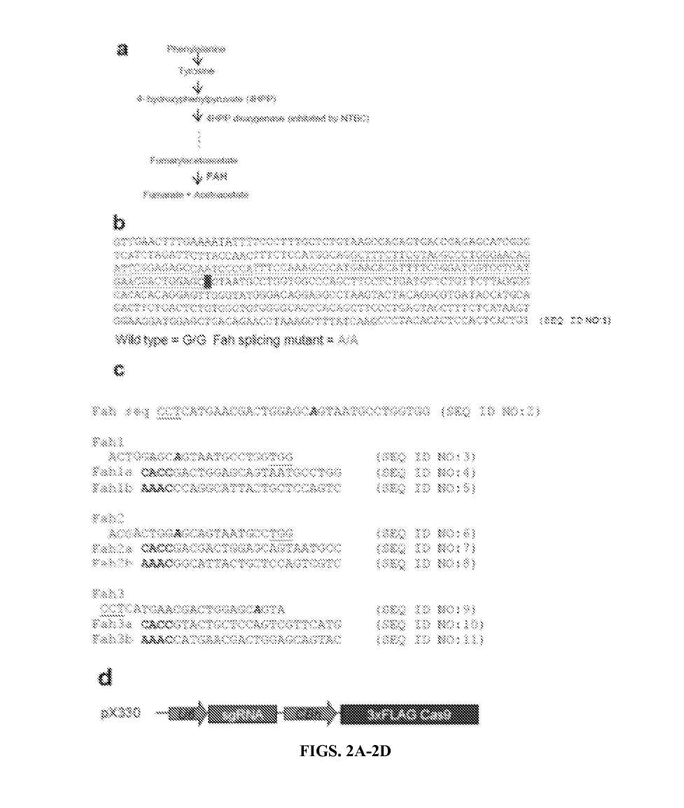

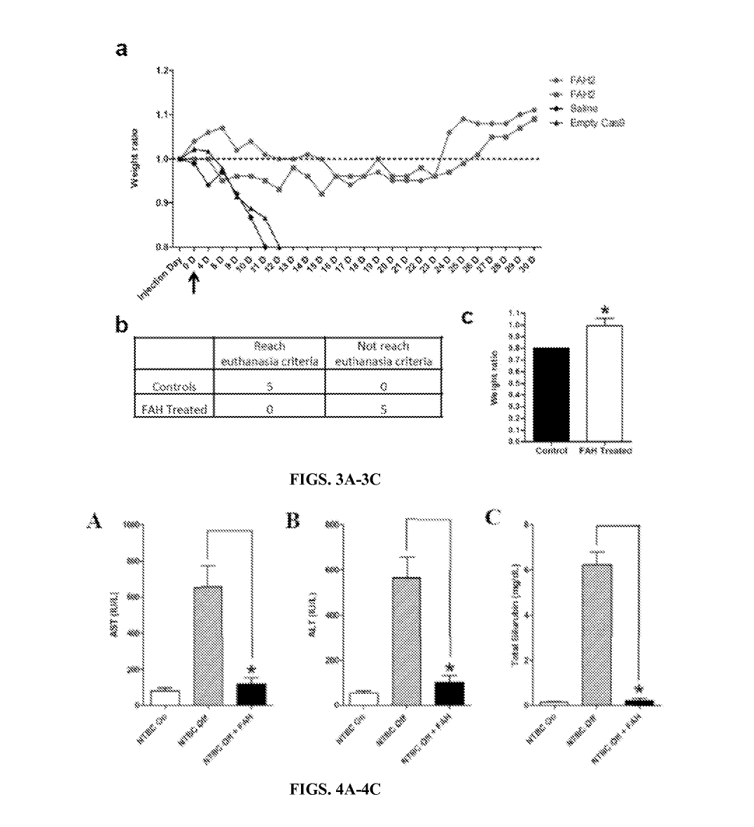

CRISPR and ssDNA were delivered to the liver in adult mice by performing hydrodynamic tail vein injection with ssDNA oligo plus pX330 (termed empty Cas9) or pX330 expressing three Fah sgRNAs (termed FAH1, FAH2, and FAH3) (Liu, et al., Gene Ther., 6, 1258-1266 (1999)). As shown in FIG. 1b and FIG. 3A-3C, Fah^{mut/mut} mice injected with saline or ssDNA oligo alone or empty Cas9 rapidly lost 20% body weight without NTBC water and had to be sacrificed. In contrast, FAH2 completely blocked weight loss; whereas the weight loss in FAH1 and FAH3 mice was less than 20% after 30 days without NTBC water. Recovery of FAH1 and FAH3 treated mice in NTBC water led to a complete rescue of body weight upon a second round of NTBC water withdrawal (FIG. 1C), indicating rescue of the Fah deficiency phenotype.

### Example 2

### CRISPR Genome Editing Generates Fah+Hepatocytes In Vivo Materials and Methods

Immunohistochemistry and Serum biochemistry

Mice were sacrificed by carbon dioxide asphyxiation. Livers were fixed with 4% paraformaldehyde (PFA), embedded in paraffin, sectioned at 5 μm and stained with hematoxylin and eosin (H&E) for pathology. Liver sections were de-waxed, rehydrated and stained using standard immunohistochemistry protocols (Xue, et al., Cancer Discov., 1, 236-247 (2011)). The following antibodies were used: anti-Fah (Abcam, 1:400), anti-FLAG (Sigma, 1:2000). The number of positive cells was quantified from >3 regions per mouse in at least 3 mice per group.

Blood was collected using retro-orbital puncture before each group of mice was sacrificed. ALT, AST and bilirubin levels in serum were determined using diagnostic assay kits (Teco Diagnostics, CA).

### Results

To examine whether CRISPR genome editing generates Fah+hepatocytes in vivo, CRISPR treated liver was stained with a Fah-specific antibody by immunohistochemistry (IHC) staining The staining reveled that FAH2 generated widespread patches of Fah+hepatocytes (33.5%±3.3%) in the Fah^{mut/mut} liver.

Concordantly, liver damage was significantly reduced compared to Fah^{mut/mut} mice off NTBC water, as indicated by improved liver histology and serum markers such as aspartate aminotransferase (AST), alanine aminotransferase (ALT), and bilirubin (FIG. 4A-4C), indicating a functional rescue of the Fah deficiency-induced liver damage.

### Example 3

### CRISPR Genome Editing Corrects the Fah Splicing Mutation in the Liver

### Materials and Methods

### Gene Expression Analysis, RT-PCR and qPCR

RNA was purified using Trizol (Invitrogen) and reverse-transcribed using a High-Capacity cDNA Reverse Transcription Kit (Applied Biosystems). Real-time PCR (qPCR) reactions were performed using gene specific primers (Applied Biosystems). Data were normalized to Actin.

### Results

To determine whether CRISPR corrects the Fah splicing mutation in the liver, reverse transcription PCR (RT-PCR) was performed using primers spanning exons 5 and 9. The results showed that wild-type mice had a 405 bp PCR band containing exon 8 and Fah^{mut/mut} mice had a 305 bp PCR band corresponding to the truncated Fah mRNA lacking exon 8. In Fah^{mut/mut} mice injected with FAH1, 2 and 3, both the 305 and 405 bp PCR bands were observed, indicating that the exon 8 to exon 9 splicing is restored in a subset of hepatocytes. Sequencing of the 405 bp bands in CRISPR-treated mice confirmed that the corrected “G” nucleotide is included in the PCR product.

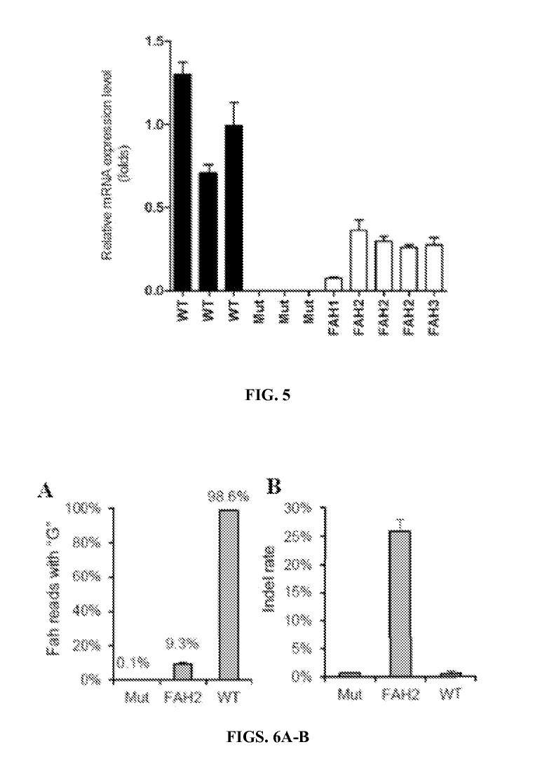

To quantitatively measure the fraction of fully spliced Fah mRNA in CRISPR-treated mice, QPCR was carried out using primers spanning exon 8 and 9. As shown in FIG. 3c, CRISPR-treated mice showed 8-36% Fah mRNA compared to wildtype mice, which is consistent with the ratio of the Fah+hepatocytes detected by IHC and the percentage of A to G correction in whole liver genomic DNA by next generation sequencing (FIGS. 5 and 6A-6B). These data support a conclusion that CRISPR corrected the exon-skipping point mutation in a subset of liver cells and generated exon 8-containing Fah mRNA in vivo.

### Example 4

### CRISPR Genome Editing is Safe and Effective

### Materials and Methods

Off-target analysis, surveyor assay and Illumina sequencing Mouse 3T3 cells were stably infected with HRasV (Li, et al., Nat Biotechnol., 31, 684-686 (2013)) to enhance transfection efficiency. 3T3 cells expressing HRas were then transiently transfected with pX330. Fah sgRNA1-3 using FugeneHD. Fah genomic region was PCR amplified. Off-target sites were predicted using the prediction tool describe in Hsu, et al., Nat Biotechnol., 31:827-832 (2013). For surveyor assay, PCR products were gel purified, treated with Suveryor nuclease kit (Transgenomic), and separated on ethidium bromide stained 4-20% Novex TBE Gels (Life Technologies).

Fah on-target and/or off-target PCR products were column purified or gel-purified (Zymo). Deep sequencing libraries were made from 1˜100 ng of the PCR products using Nextera protocol (Illumina). Libraries were normalized to approximately equal molar ratio, and sequenced on Illumina MiSeq machines (150 bp, paired-end). Reads were mapped to the PCR amplicons as references using bwa with custom scripts. Data processing was performed according to standard Illumina sequencing analysis procedures.

### Statistics

P values were determined by Fisher's exact test, Student's t-tests and One-Way ANOVA using Prism 5 (GraphPad).

### Results

To characterize potential off-target effects of CRISPR, a published prediction tool (Hsu, et al., Nat Biotechnol., 31, 827-832 (2013)) was used to identify potential FAH off-target genomic sites in the mouse genome. In mouse 3T3 cells transfected with FAH or control, the CRISPR-mediated editing at the Fah locus and potential sites was measured using the Surveyor assay (Cong, L. et al., Science, 339, 819-823 (2013)). Surveyor cutting was detected at the Fah site, indicating that the one nucleotide mismatch between FAH1-3 and the wildtype Fah gene allows CRISPR editing. Surveyor cutting was not detected at the assayed off-target sites with multiple mismatches. The PCR products from off-target sites of FAH2 were sequenced, and minimal indels were detected.

To investigate the safety of CRISPR in mice, a cohort of wildtype FVB mice were injected with empty Cas9 plasmid or a Cas9 plus a sgRNA targeting GFP via hydrodynamic injection. Three months later, the Cas9 or Cas9/sgRNA mice were indistinguishable with respect to body weight compared to saline controls (FIG. 7A). Histopathological analysis revealed neither obvious pathological changes in the liver nor any signs of hyperplasia (FIG. 7B). These data indicate that transient expression of CRISPR/Cas9 in the liver is well-tolerated in mice, an observation which is consistent with a recent study showing long-term Cas9/sgRNA expression is not toxic in cells (Malina, et al., Genes Dev., 27, 2602-2614 (2013)).

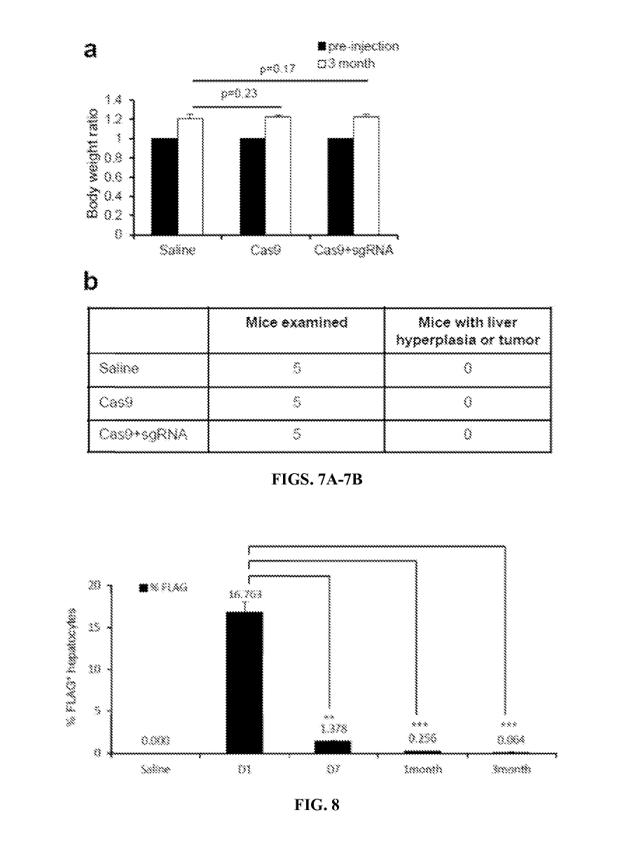

To examine the rate of potential CRISPR plasmid DNA integration and expression in the liver, pX330 plasmids were injected into a cohort of wildtype FVB mice and the expression of FLAG tagged Cas9 was measured by IHC staining using a FLAG tag antibody. As shown in FIG. 8, an average of 16.78% FLAG positive hepatocytes at one day post injection was detected. In contrast, FLAG IHC staining was detected in 0.26+0.06% and 0.06+0.11% of hepatocytes after 1 month and 3 months post injection, respectively. These data indicate that integration of vector DNA is minimal in the liver.

To measure the initial Fah gene repair frequency, Fah^{mut/mut th}mice were treated with FAH2 and maintained on the NTBC water for 6 days. Fah positive cells were determined by IHC staining. Initial repair frequency without hepatectomy was determined to be 0.40%±0.12%.

In summary, the Examples collectively demonstrate the potential to correct disease genes in adult mouse liver using a CRISPR/Cas system in vivo. Transient expression of Cas9, sgRNA and a co-injected ssDNA by non-viral hydrodynamic injection is sufficient to rescue the weight loss of Fah^{mut/mut} mice, a model of hereditary tyrosinemia type I in humans. In vivo delivery of CRISPR/Cas system generated Fah+hepatocytes and corrected the splicing point mutation. The data indicate that CRISPR is capable of gene correction in adult mouse liver, extending the potential application of CRISPR from in vitro study to in vivo genome editing in adult mammalian models. The strong positive selection and expansion of Fah+hepatocytes in the Fah^{mut/mut} liver may have contributed to the correction of Fah mutation in this mouse model (Faulk, et al., Hepatology, 51, 1200-1208 (2010)). Some studies advice that improvement to the CRISPR delivery technique to increase the rate of gene correction is needed for other disease models (Li, et al., Nature, 475, 217-221 (2011)). FAH2 also introduced indels at the predicted Cas9 cutting site, which is consistent with the literature that Cas9 induced DSB is repaired by both NHEJ and HDR when ssDNA is provided (Cong, L. et al., Science, 339, 819-823 (2013)). In the Fah^{mut/mut} mice, such NHEJ events are unlikely causing phenotype because unrepaired Fah mRNA is not stable. Using Cas9 nickase may help to ensure precise gene repair (Ran, et al., Cell, 154, 1380-1389 (2013)) and reduce the potential for off-target mutagenesis (Fu, et al., Nat. Biotechnol., 31, 822-826 (2013)). In conclusion, this study indicates that therapeutic correction of genetic disease with CRISPR is possible.

1. A method of transfecting cells in vivo comprising administering to a subject an injectable pharmaceutical composition comprising a genome editing composition comprising one or more nucleic acid constructs that can express the elements of a CRISPR/Cas system, a zinc finger nuclease, or transcription activator-like effector nuclease (TALEN) in a transfected cell, and a pharmaceutically acceptable carrier, by hydrodynamic injection into a vessel of the subject,
wherein the pharmaceutical composition is administered in a volume and at rate of injection suitable to transfect target eukaryotic cells in the subject with an effective amount of the genome editing composition to alter the genome of the target cells.

2. The method of claim 1 wherein the nucleic acid constructs that can express the elements of the CRISPR/Cas-mediated system are one or more plasmids encoding (a) a chimeric RNA (chiRNA) polynucleotide sequence, wherein the polynucleotide sequence includes (i) a guide sequence capable of hybridizing to a genomic target sequence in the target cells, (ii) a tracr mate sequence, and (iii) a tracr sequence; and (b) an enzyme-coding sequence encoding a CRISPR enzyme, wherein (a) and (b) are operably linked to the same or different promoters capable of driving expression of (a) and (b) in the target cells in an amount effective to induce a single or double strand break at a target site in the genome of the target cells.

3. The method of claim 1 wherein the genome editing composition further comprises a donor polynucleotide suitable for recombination into the genome of the target cells at or adjacent to the target site.

4. The method of claim 3 wherein the donor polynucleotide introduces one or more insertions, deletions, or substitution in the target cells' genome.

5. The method of claim 4 wherein the substitution corrects a point mutation.

6. The method of claim 5 wherein the point mutation is associated with genetic disease or condition.

7. The method of claim 1 wherein the target cells are liver cells, spleen cells, heart cells, kidney cells, lung cells, skeletal muscle cells (myofiber, myocytes) bone cells (osteocytes, osteoclasts, osteoblasts), bone marrow cells, stroma cells, joint cells (synovial and cartilage cells), connective tissue cells (fibroblasts, fibrocytes, chondrocytes, mesenchyme cells, mast cells, macrophages, histiocytes), cells in tendons, cells in the skin, or cells in the lymph nodes.

8. The method of claim 7 wherein the target cells are liver cells.

9. The method of claim 1 wherein the hydrodynamic injection results in systemic circulation of the injectable pharmaceutical composition.

10. The method of claim 1 wherein the hydrodynamic injection results in region or local, but not systemic circulation of the injectable pharmaceutical composition.

11. The method of claim 10 further comprising occluding one or more vessels of the subject to direct the flow of the pharmaceutical composition toward the target cells.

12. The method of claim 1 wherein the vessel is selected from the group consisting of tail vein, tail artery, inferior vena cava, superior vena cava, jugular vein, hepatic vein, hepatic artery, portal vein, bile duct, saphenous, cephalic and median veins, femoral vein, femoral artery, brachial and popliteal arteries, iliac arteries, renal vein, carotid artery, and aorta.

13. A method of a treating a subject for genetic disease comprising transfecting an effective number of target cells of the subject according the method of claim 1 to reduce or prevent one or more symptoms of the disease.

14. The method of claim 13 wherein the genetic disease is caused by a point mutation in target cells' genome.

15. The method of claim 14 wherein the point mutation is in a promoter, or gene intron or exon.

16. The method of claim 15 wherein the point mutation causes aberrant transcription of a gene in the target cells.

17. The method of claim 15 wherein the point mutation causes translation of a mutated protein in the subject.

18. The method of claim 13 is one characterized by positive selection, wherein alteration of the genome of 1%-75%, or 10%-50%, or 20%-40% of the target cells is effective to treat the disease or condition.

19. The method of claim 13 wherein the target cells are hepatocytes.

20. The method of claim 19 wherein the disease or condition is hereditary tyrosinemia type I (HTI).


SEQUENCE LISTING (12 sequences)

SEQ ID NO: 1 - DNA, 420 bp, Mus musculus
gttgaactttgaaaatattttccctttgctctgtaagccacagtgacccagagcatcgggtcatctagattcttaccaactttctccatggcaggctttcttcgtaggccctgggaacagattcggagagccaatccccatttccaaagcccatgaacacattttcgggatggtcctcatgaacgactggagcagtaatgcctggtggcccagcttcctctgatgttctgttcttaggggcacacacaggagttgggtatgggacaggaggcctaagtactacaggggtgataccatgcagacttctgactctgtgggtgtggggcagtcacagcttccctgagtagctttctcataagtggaaggatggagctgacagaacctaaagctttatcaagccctacacactccactcactgt

SEQ ID NO: 2 - DNA, 34 bp, Artificial Sequence
  FAH subgenomic RNA
cctcatgaacgactggagcagtaatgcctggtgg

SEQ ID NO: 3 - DNA, 23 bp, Artificial Sequence
  FAH subgenomic RNA
actggagcagtaatgcctggtgg

SEQ ID NO: 4 - DNA, 25 bp, Artificial Sequence
  FAH subgenomic RNA
caccgactggagcagtaatgcctgg

SEQ ID NO: 5 - DNA, 25 bp, Artificial Sequence
  FAH subgenomic RNA
aaacccaggcattactgctccagtc

SEQ ID NO: 6 - DNA, 23 bp, Artificial Sequence
  FAH subgenomic RNA
acgactggagcagtaatgcctgg

SEQ ID NO: 7 - DNA, 25 bp, Artificial Sequence
  FAH subgenomic RNA
caccgacgactggagcagtaatgcc

SEQ ID NO: 8 - DNA, 25 bp, Artificial Sequence
  FAH subgenomic RNA
aaacggcattactgctccagtcgtc

SEQ ID NO: 9 - DNA, 23 bp, Artificial Sequence
  FAH subgenomic RNA
cctcatgaacgactggagcagta

SEQ ID NO: 10 - DNA, 25 bp, Artificial Sequence
  FAH subgenomic RNA
caccgtactgctccagtcgttcatg

SEQ ID NO: 11 - DNA, 25 bp, Artificial Sequence
  FAH subgenomic RNA
aaaccatgaacgactggagcagtac

SEQ ID NO: 12 - DNA, 199 bp, Artificial Sequence
  Synthetic Oligomer
gctttcttcgtaggccctgggaacagattcggagagccaatccccatttccaaagcccatgaacacattttcgggatggtcctcatgaacgactggagcggtaatgcctggtggcccagcttcctctgatgttctgttcttaggggcacacacaggagttgggtatgggacaggaggcctaagtactacaggggtgata